(19) United States
(12) Patent Application Publication
(10) Pub. No.: US 2015/0282708 A1
(43) Pub. Date: Oct. 8, 2015

(54) SYSTEMS AND METHODS FOR A MEDICAL CONNECTOR ENABLING WIRELESS COMMUNICATIONS

(71) Applicant: Covidien LP, Mansfield, MA (US)

(72) Inventors: Friso Schlottau, Lyons, CO (US); Henry J. Szymanski, Broomfield, CO (US); Tony C. Carnes, Gainesville, FL (US)

(21) Appl. No.: 14/680,499

(22) Filed: Apr. 7, 2015

Related U.S. Application Data
(60) Provisional application No. 61/976,760, filed on Apr. 8, 2014.

(51) Int. Cl. A61B 5/00; A61B 5/1455

(52) U.S. Cl. CPC A61B 5/0022; A61B 5/14552

(57) ABSTRACT

The present embodiments relate to a patient monitoring system having a patient monitor and a wireless pre-amplifier in wireless communication with the patient monitor. The wireless pre-amplifier is configured to receive a signal related to a physiological parameter of a patient from one or more sensors applied to the patient. The wireless pre-amplifier also includes a wireless attachment configured to wirelessly transmit the signal related to the physiological parameter of the patient to the patient monitor over a wireless communications channel, and an indicator feature configured to provide a user perceptible indication related to the wireless communication channel.

### CROSS-REFERENCE TO RELATED APPLICATION

This application claims priority to and the benefit of U.S. Provisional Application No. 61/976,760, entitled “SYSTEMS AND METHODS FOR A MEDICAL CONNECTOR ENABLING WIRELESS COMMUNICATIONS”, filed Apr. 8, 2014, which is herein incorporated by reference in its entirety.

### BACKGROUND

The present disclosure relates generally to patient monitoring systems and, more particularly, to a wireless medical connector retrofitted to medical devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for non-invasively monitoring many such physiological characteristics. These devices provide doctors and other healthcare personnel with the information they need to provide the best possible healthcare for their patients. As a result, such monitoring devices have become an indispensable part of modern medicine.

A wide variety of devices have been developed for non-invasively monitoring physiological characteristics of patients. For example, an oximetry sensor system may non-invasively detect various patient blood flow characteristics, such as the blood-oxygen saturation of hemoglobin in blood, the volume of individual blood pulsations supplying the tissue, and/or the rate of blood pulsations corresponding to each heart beat of a patient. During operation, the oximeter sensor emits light and photoelectrically senses the absorption and/or scattering of the light after passage through the perfused tissue. A photo-plethysmographic waveform, which corresponds to the cyclic attenuation of optical energy through the patient's tissue, may be generated from the detected light. Additionally, one or more physiological characteristics may be calculated based upon the amount of light absorbed or scattered. More specifically, the light passed through the tissue may be selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

For example, a reflectance-type sensor placed on a patient's forehead may emit light into the site and detect the light that is “reflected” back after being transmitted through the forehead region. The amount of light detected may provide information that corresponds to valuable physiological patient data. The data collected by the sensor may be used to calculate one or more of the above physiological characteristics based upon the absorption or scattering of the light. For instance, the emitted light is typically selected to be of one or more wavelengths that are absorbed or scattered in an amount related to the presence of oxygenated versus de-oxygenated hemoglobin in the blood. The amount of light absorbed and/or scattered may be used to estimate the amount of the oxygen in the tissue using various algorithms.

The sensors generally include one or more emitters that emit the light and one or more detectors that detect the light. The emitters and detectors may be housed in a reusable or disposable oximeter sensor that couples to the oximeter electronics and the display unit (hereinafter referred to as the monitor). The monitor may collect historical physiological data for the patient, which may be used by a clinician or medical personnel for diagnostic and monitoring purposes. Patients are often moved to various locations during treatment. For example, a patient may be transported in an ambulance, delivered to an emergency room, moved to an operating room, transferred to a surgical recovery room, transferred to an intensive care unit, and then moved to a nursing floor or other locations. Thus, the patient may be moved between various locations within the same hospital, or between different hospitals. The sensor employed to monitor the condition of the patient may be adhesive in its attachment and remain with the patient. The monitors, however, may be local to particular locations within a facility or vehicle. Thus, the sensor may be disconnected from the monitor at a departure site and reconnected to another monitor at a destination site. Consequently, patient-related data (e.g., historical physiological data) collected by the monitor at the departure site may be unavailable to the clinician attending the patient at the destination site.

Such patient sensors may communicate with a patient monitor using a communication cable. For example, a sensor may use such a communication cable to send a signal, corresponding to a measurement performed by the sensor, to the patient monitor for processing. However, the use of cables may limit the range of applications available, as the cables may become prohibitively expensive at long distances as well as limit a patient's range of motion by physically tethering the patient to a monitoring device. As such, it may be desirable to monitor the physiological parameters of a patient with wireless sensors. However, in some situations, it may be difficult to add wireless capabilities to existing medical devices that do not already have wireless capabilities without modifying or altering the design of existing hardware. Further, in some embodiments, sensors are typically paired with a patient monitor to ensure that the patient monitor is displaying information from the intended source. Accordingly, it may be desirable to safely and accurately identify which sensor is providing a signal, corresponding to a physiological measurement, to the patient monitor. Further, it may be desirable to safely and accurately indicate the quality and/or the status of the signal between the wireless sensor and the patient monitor.

### BRIEF DESCRIPTION OF THE DRAWINGS

Advantages of the disclosed techniques may become apparent upon reading the following detailed description and upon reference to the drawings in which:

FIG. 1 is a front view of an embodiment of a monitoring system configured to be used with a wireless pre-amplifier and a sensor for regional saturation, in accordance with an aspect of the present disclosure;

FIG. 2 is a block diagram of an embodiment of the monitoring system of FIG. 1, in accordance with an aspect of the present disclosure;

FIG. 3 is a perspective view of an embodiment of the monitoring system of FIG. 1, illustrating the wireless pre-amplifier and the sensor as applied to a patient's forehead;

FIG. 4 is a perspective view of an embodiment of the monitoring system of FIG. 1, illustrating the wireless pre-amplifier and the sensor coupled to an ear-piece attached to a patient's ear;

FIG. 5 is a perspective view of an embodiment of the monitoring system of FIG. 1, illustrating the wireless pre-amplifier and the sensor disposed proximate to the patient and remote from the patient monitor; and

FIG. 6 is a block diagram of an embodiment of the monitoring system of FIG. 1, illustrating a charging/linking station configured to charge and/or pair the wireless pre-amplifier with the sensor.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Also, as used herein, the term “over” or “above” refers to a component location on a sensor that is closer to patient tissue when the sensor is applied to the patient.

Wireless pre-amplifiers (e.g., configured to receive and amplify, filter, and/or digitize the signals collected by the sensors) coupled to one or more sensors may be used to provide a patient with a greater freedom of movement when compared to wired pre-amplifiers coupled to a patient monitor. In certain circumstances, it may be desirable to have a wireless pre-amplifier and a patient monitor functioning together to monitor one or more physiological parameters of the patient. However, in some situations, it may be difficult to add wireless capabilities to existing medical devices that do not already have means for wireless communications. For example, it may be difficult to add wireless communication capabilities to existing patient monitors or pre-amplifiers devices without modifying or altering the design of existing hardware. Accordingly, it may be desirable to provide systems and methods configured to incorporate wireless communication into a medical system via wireless attachments without modifying or altering the design of existing hardware. Further, it may be desirable to provide for systems and methods for pairing and/or linking the wireless attachments so that the patient monitor receives physiological parameter information from the intended source.

With the forgoing in mind, the present embodiments relate to a wireless pre-amplifier configured to receive one or more physiological characteristics of a patient, such as regional oxygen saturation (rSO₂) information from one or more sensors. In particular, the wireless pre-amplifier may be enabled with wireless connectivity between one or more components of the patient monitoring system, such as the patient monitor. For example, in certain embodiments, the wireless pre-amplifier may include a wireless module configured to wirelessly communicate with a wireless module of the patient monitor. In certain embodiments, a wireless attachment may be attached to the pre-amplifier and/or the patient monitor to enable wireless connectivity. Accordingly, the pre-amplifier may be configured to transmit either raw detector signals or detector signals received from the one or more sensors that have been amplified, filtered, and digitized to the patient monitor over a wireless communications channel.

In certain embodiments, the wireless attachment may be an electrical connector configured with wireless capabilities and one or more indicator features. For example, the connector may be a serial connector (e.g., a Fischer connector, a S-pin male/female connector, a 15-pin male/female connector, a 24-pin male/female connector, a 26-pin male/female connector, a medi-snap connector, etc.) that may be configured to replace a wired connection between two or more components of the medical device, and may be used to establish one or more wireless communication channels between the system components. For example, the connector may replace a wired connection between a patient monitor and a wireless pre-amplifier, between a patient monitor and a multi-parameter computing device, between two or more patient monitors and a wireless pre-amplifier, and so forth. In some embodiments, a indicator (e.g., alarms, voice warnings, beep tones, LEDs, different flashing patterns of LEDs, different LED colors, color changing bands, color patterns or schemes, etc.) may be used to indicate a coupling or connectivity status (e.g., coupled, unable to be coupled, etc.), a wireless signal or quality status (e.g., signal strength, signal lost, etc.), and so forth.

The present techniques may be used in conjunction with any type of displayed medical data. For example, the medical data may be collected using a particular sensor or set of sensors, such as regional oxygen saturation sensors. By way of example, an INVOS® cerebral/somatic sensor, such as an OxyAlert™ NIR sensor by Covidien LP or a SomaSensor® by Covidien LP, which may include one or more emitters and a pair of detectors for determining site-specific oxygen levels, may represent such sensors. Particularly, the present techniques may be used to transmit the collected medical data between components of the device instead of the communication cable typically used. The present techniques may also be used in conjunction with other types of medical sensors, such as pulse oximetry sensors or carbon dioxide sensors.

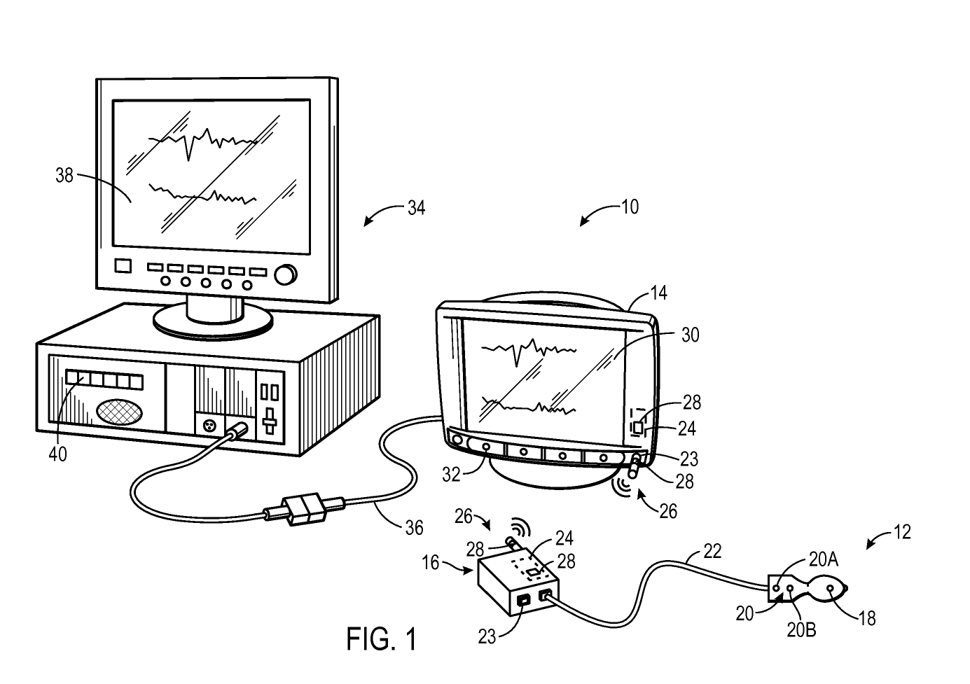

With this in mind, FIG. 1 depicts an embodiment of a patient monitoring system 10 that may be used in conjunction with a medical sensor 12, a patient monitor 14, and a wireless pre-amplifier 16. Although the depicted embodiments relate to sensors for use on a patient's head, it should be understood that, in certain embodiments, the features of the sensor 12 as provided herein may be incorporated into sensors for use on other tissue locations, such as the back, the stomach, the heel, the ear, an arm, a leg, or any other appropriate measurement site. In addition, although the embodiment of the patient monitoring system 10 illustrated in FIG. 1 relates to photoplethysmography or regional oximetry, the system 10 may be configured to obtain a variety of medical measurements with a suitable medical sensor. For example, the system 10 may additionally be configured to determine patient electroencephalography (e.g., a bispectral index), or any other desired physiological parameter such as water fraction or hematocrit.

As noted above, the system 10 includes the sensor 12 that is communicatively coupled to the wireless pre-amplifier 16. Although only one sensor 12 is shown coupled to the wireless pre-amplifier 16 in FIG. 1, in other embodiments, two, three, four, or more sensors 12 may be coupled to the wireless pre-amplifier 16. For example, two sensors 12 may be used for cerebral oximetry and simultaneously two other sensors 12 used for somatic oximetry. Thus, multiple sensors 12 may utilize a single pre-amplifier 16. As shown in FIG. 1, the sensor 12 includes an emitter 18 and a pair of detectors 20. The emitter 18 and detectors 20 of the sensor 12 are coupled to the wireless pre-amplifier 16 via a cable 22. The cable 22 may interface directly with the sensor 12 and may include a plurality of conductors (e.g., wires). In certain embodiments, the sensor 12 may be configured to store patient-related data, such as historical regional oximetry data (e.g., rSO₂ information).

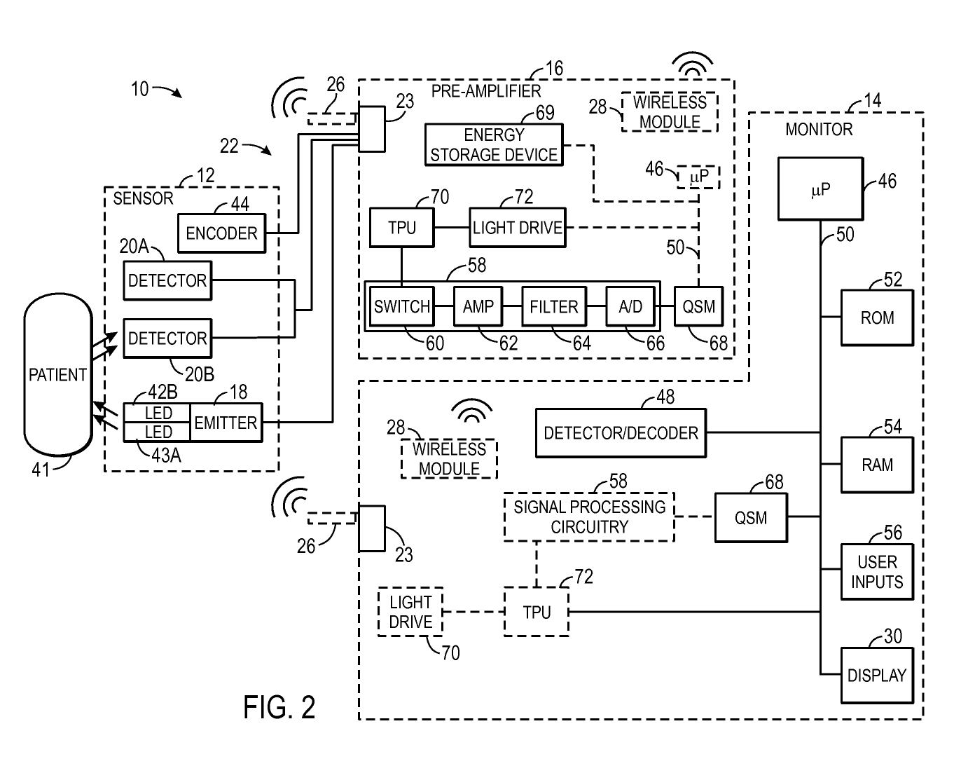

In certain embodiments, the wireless pre-amplifier 16 of the system 10 is configured to receive the signals collected by the detectors 20 via the cable 22 at one or more input ports 23. The pre-amplifier 16 may be configured for amplifying, filtering, and digitizing the electrical signals received from the detectors 20, as further described with respect to FIG. 2. In certain embodiments, the pre-amplifier 16 may be provided as a part of the patient monitor 14. Further, in certain embodiments, the pre-amplifier 16 may be external to the patient monitor 14, and may be coupled to the monitor 14 via one or more additional cables (not shown). In the illustrated embodiments, the wireless pre-amplifier 16 is disposed external to the patient monitor 14, and may be configured to wirelessly communicate with the patient monitor 14.

For example, in some embodiments, the pre-amplifier 16 may include a wireless module 24 (e.g., transceiver including a transmitter and/or receiver) for transmitting and receiving wireless data, a memory, and various other amplifying, filtering, and/or digitizing structures. The wireless module 24 of the pre-amplifier 16 may be configured to may establish wireless communication (e.g., wireless communication data and/or authentication channels) with the wireless module 24 of the patient monitor 14 using any suitable protocol. For example, the wireless modules 24 may be capable of communicating using the IEEE 802.15.4 standard, and may be, for example, ZigBee, WirelessHART, or MiWi modules. Additionally or alternatively, the wireless modules 24 may be capable of communicating using the Bluetooth standard, one or more of the IEEE 802.11 standards, an ultra-wideband (UWB) standard, or a near-field communication (NFC) standard. In certain embodiments, the wireless module 24 of the wireless pre-amplifier 16 may be used to transmit either raw detector signals or detector signals that have been amplified, filtered, and digitized to the patient monitor 14 for further processing and/or analysis, as further explained with respect to FIG. 2.

In some embodiments, the pre-amplifier 16 may establish a wireless communication with the patient monitor 14 via one or more wireless attachments 26. Specifically, the wireless attachment 26 may be an electrical connector configured with wireless capabilities and coupled or attached to components of existing medical systems 10 to replace wired connections between two or more components of the system 10, such as between the patient monitor 14 and the multi-parameter patient monitor 34, between the patient monitor 14 and the pre-amplifier 16, between the pre-amplifier 16 and the multi-parameter patient monitor 34, etc. The electrical connector may be a serial connector (e.g., a 9-pin male/female connector, a 15-pin male/female connector, a 24-pin male/female connector, a 26-pin male/female connector, a medi-snap connector, etc.) configured with wireless capabilities. The wireless attachment 26 may be electrically and/or physically connected to the pre-amplifier 16 via the input ports 23. In certain embodiments, the wireless attachment 26 of the pre-amplifier 16 may be configured to establish wireless communication (e.g., wireless communication data and/or authentication channels) with the wireless attachment 26 of the patient monitor 14, using any suitable protocol, such as those described above. In certain embodiments, the wireless attachment 26 of the wireless pre-amplifier 16 may be used to transmit either raw detector signals or detector signals that have been amplified, filtered, and digitized to the patient monitor 14 for further processing and/or analysis, as further explained with respect to FIG. 2. The wireless attachment 26 may be electrically and/or physically coupled to the patient monitor 14 at one or more patient monitor 14 input ports 23. It should be noted that in certain embodiments, the wireless modules 24 and/or the wireless attachments 26 of the patient monitor 14 and the wireless pre-amplifier 16 may be utilized in any combination. For example, either the wireless module 24 or the wireless attachment 26 of the pre-amplifier 16 may establish wireless communication with either the wireless attachment 26 or the wireless module 24 of the patient monitor 14. Accordingly, in certain embodiments, an indicator feature 28 on the wireless modules 24 or the wireless attachments 26 may be utilized by the system 10 and/or an operator to indicate the pairing and/or the status of connectivity between the pre-amplifier 16 and the patient monitor 14. It should be noted that in some embodiments, the wireless module 24 may be a preexisting wireless capability incorporated into a component of the system 10. In other embodiments, the wireless module 24 may be the wireless attachment 26 permanently or temporarily fixed to the component of the system 10.

The monitor 14 includes a monitor display 30 configured to display information regarding the physiological parameters monitored by the sensor 12, information about the system, and/or alarm indications. The monitor 14 may include various input components 32, such as knobs, switches, keys and keypads, buttons, etc., to provide for operation and configuration of the monitor 14. The monitor 14 also includes a processor that may be used to execute code, such as code for implementing various monitoring functionalities enabled by the sensor 12. In certain embodiments, for example, the monitor 14 may be configured to process raw signals generated by the detectors 20 to estimate the amount of oxygenated vs. de-oxygenated hemoglobin in a monitored region of the patient. In some embodiments, as discussed in further detail with respect to FIG. 2, the monitor 14 may be configured to receive processed signals generated by the pre-amplifier 16 to estimate the amount of oxygenated vs. de-oxygenated hemoglobin in a monitored region of the patient.

The monitor 14 may be any suitable monitor, such as an INVOS® System monitor available from Covidien LP. Furthermore, to upgrade conventional operation provided by the monitor 14 to provide additional functions, the monitor 14 may be coupled to a multi-parameter patient monitor 34 via a cable 36 connected to a sensor input port. In addition to the monitor 14, or alternatively, the multi-parameter patient monitor 34 may be configured to calculate physiological parameters and to provide a central display 38 for the visualization of information from the monitor 14 and from other medical monitoring devices or systems. The multi-parameter monitor 34 includes a processor and a memory that may be configured to execute and store code. The multi-parameter monitor 34 may also include various input components 40, such as knobs, switches, keys and keypads, buttons, etc., to provide for operation and configuration of the multi-parameter monitor 34. In addition, the monitor 14 and/or the multi-parameter monitor 34 may be connected to a network to enable the sharing of information with servers or other workstations.

As provided herein, the sensor 12 may be configured to perform regional oximetry. Indeed, in one embodiment, the sensor 12 may be an INVOS® cerebral/somatic sensor available from Covidien LP. In regional oximetry, by comparing the relative intensities of light received at two or more detectors, it is possible to estimate the blood oxygen saturation of hemoglobin in a region of a body. For example, a regional oximeter may include a sensor to be placed on a patient's forehead and may be used to calculate the oxygen saturation of a patient's blood within the venous, arterial, and capillary systems of a region underlying the patient's forehead (e.g., in the cerebral cortex). As illustrated in FIGS. 1 and 2, the sensor 12 may include the emitter 18 and the two detectors 20: one detector 20A that is relatively “close” (i.e., proximal) to the emitter 18 and another detector 20B that is relatively “far” (i.e., distal) from the emitter 18. Light intensity of one or more wavelengths may be received at both the “close” and the “far” detectors 20A and 20B. Thus, the detector 20A may receive a first portion of light and the detector 20B may receive a second portion of light. Each of the detectors 20 may generate signals indicative of their respective portions of light. For example, the resulting signals may arrive at a regional saturation value that pertains to additional tissue through which the light received at the “far” detector 20B passed (tissue in addition to the tissue through which the light received by the “close” detector 20A passed, e.g., the brain tissue) when it was transmitted through a region of a patient (e.g., a patient's cranium). Surface data from the skin and skull is subtracted out to produce a regional oxygen saturation (rSO₂) value for deeper tissues.

In particular, the pre-amplifier 16 may be configured for amplifying, filtering, and digitizing the electrical signals received from the detectors 20, as further described with respect to FIG. 2, illustrating a simplified block diagram of the medical system 10 in accordance with an embodiment. The sensor 12 may include optical components in the forms of emitters 18 and detectors 20. The emitter 18 and the detector 20 may be arranged in a reflectance or transmission-type configuration with respect to one another. However, in embodiments in which the sensor 12 is configured for use on a forehead of a patient 41, the emitters 18 and detectors 20 may be in a reflectance configuration. The emitter 18 may also be a light emitting diode, superluminescent light emitting diode, a laser diode or a vertical cavity surface emitting laser (VCSEL). The emitter 18 and detector 20 may also include optical fiber sensing elements. The emitter 18 may include a broadband or “white light” source, in which case the detector could include any of a variety of elements for selecting specific wavelengths, such as reflective or refractive elements or interferometers. These kinds of emitters and/or detectors would typically be coupled to the sensor 12 via fiber optics. Alternatively, a sensor assembly 10 may sense light detected from the tissue is at a different wavelength from the light emitted into the tissue. Such sensors may be adapted to sense fluorescence, phosphorescence, Raman scattering, Rayleigh scattering and multi-photon events or photoacoustic effects. In one embodiment, the emitter 18 may be configured for use in a regional saturation technique. To that end, the emitter 18 may include two light emitting diodes (LEDs) 42A and 42B that are capable of emitting at least two wavelengths of light, e.g., red or near infrared light. In one embodiment, the LEDs emit light in the range of about 600 nanometers to about 1000 nm. In a particular embodiment, the one LED 40 is configured to emit light at about 730 nm and the other LED is configured to emit light at about 810 nm.

In any suitable configuration of the sensor 12, the detectors 20A and 20B may be an array of detector elements that may be capable of detecting light at various intensities and wavelengths. In one embodiment, light enters the detector 20 (e.g., detector 20A or 20B) after passing through the tissue of the patient 41. In another embodiment, light emitted from the emitter 16 may be reflected by elements in the patent's tissue to enter the detector 20. The detector 20 may convert the received light at a given intensity, which may be directly related to the absorbance and/or reflectance of light in the tissue of the patient 41, into an electrical signal. That is, when more light at a certain wavelength is absorbed, less light of that wavelength is typically received from the tissue by the detector 20, and when more light at a certain wavelength is reflected, more light of that wavelength is typically received from the tissue by the detector 20. In certain embodiments, after converting the received light to an electrical signal, the detector 20 may send the signal to the monitor 14, where physiological characteristics may be calculated based at least in part on the absorption and/or reflection of light by the tissue of the patient 41. In the illustrated embodiment, after converting the received light to an electrical signal, the detectors 20 may send the signals to the pre-amplifier 16, where the signals may be amplified, filtered, and/or digitized and then wireless communicated to the monitor 14. In such embodiments, the monitor 14 may then be configured to calculate the physiological characteristics.

In certain embodiments, the medical sensor 12 may also include an encoder 44 that may provide signals indicative of the wavelength of one or more light sources of the emitter 16, which may allow for selection of appropriate calibration coefficients for calculating a physical parameter such as blood oxygen saturation. The encoder 44 may, for instance, be a coded resistor, EEPROM or other coding devices (such as a capacitor, inductor, PROM, RFID, parallel resident currents, or a colorimetric indicator) that may provide a signal to a microprocessor 46 disposed within the patient monitor 14 or the pre-amplifier 16 related to the characteristics of the medical sensor 12 to enable the microprocessor 46 to determine the appropriate calibration characteristics of the medical sensor 12. Further, the encoder 44 may include encryption coding that prevents a disposable part of the medical sensor 12 from being recognized by a microprocessor 46 unable to decode the encryption. For example, a detector/decoder 48 may translate information from the encoder 44 before it can be properly handled by the processor 46. In some embodiments, the encoder 44 may communicate with the detector/decoder 48 and/or the microprocessor 46 disposed within the patient monitor 14 via the wireless pre-amplifier 16. In some embodiments, the encoder 44 and/or the detector/decoder 48 may not be present. In certain embodiments, the signals from the detector 20 and/or the encoder 44 may be transmitted to the pre-amplifier 16 via the wired cables 22 and to the monitor 14 via the wireless communications established between the wireless pre-amplifier 16 and the monitor 14. The monitor 14 may include one or more processors 46 coupled to an internal bus 50. Also connected to the bus may be a ROM memory 52, a RAM memory 54, user inputs 56, and the display 30.

In certain embodiments, the raw signals from the detector 20 may be transmitted to the pre-amplifier 16 via the cable 22, and may be received at one or more input ports 23 on the pre-amplifier 16. Further, the wireless pre-amplifier 16 may transmit either the raw detector signals or amplified, filtered, and digitized detector signals to the patient monitor 14 via a wired connection or via the wireless communications channels established between the monitor 14 and the pre-amplifier 16. For example, as noted above, the wireless modules 28 may be disposed within the pre-amplifier 16 and/or the patient monitor 14, and the wireless attachments 26 may be coupled to the pre-amplifier 16 and/or the patient monitor 14 at the input ports 23. The wireless modules 28 and/or the wireless attachments 26 may be utilized by the pre-amplifier 16 and the patient monitor 14 in various combinations to establish a wireless communications channel. For example, in certain embodiments, the wireless attachment 23 on the patient monitor 14 may be in wireless communication with the wireless module 28 of the pre-amplifier 16. Further, in some embodiments, it should be noted that the pre-amplifier 16 and the patient monitor 14 may communicate via a wired connection, such as a cable.

In some embodiments, the pre-amplifier 16 may include a signal processing circuitry 58 comprising a switching circuitry 60, an amplifier 62, a filter 64 (e.g., a low pass filter), an analog-to-digital converter 66 (e.g., A/D), and a QSM 68. In addition, in certain embodiments, the pre-amplifier 16 may include a time processing unit (TPU) 70 configured to provide timing control signals to light drive circuitry 72. The light drive 72 may control when the emitter 16 is activated, and if multiple light sources are used, the multiplexed timing for the different light sources. In certain embodiments, the pre-amplifier 16 may include a processor 46 coupled to the signal processing circuitry 58, the TPU 70, and/or the light drive 72 via the bus 50. It is envisioned that the emitters 16 may be controlled via time division multiplexing of the light sources. The TPU 70 may also control the gating-in of signals from detector 20 through the switch 60. These signals are sampled at the proper time, depending at least in part upon which of multiple light sources is activated, if multiple light sources are used. The received signal from the detector 20 may be passed through the amplifier 62, the filter 64, and the analog-to-digital converter 66 for amplifying, filtering, and digitizing the electrical signals. The digital data may then be stored in a queued serial module (QSM) 68, for later transmission to the patient monitor 14, such as when wireless communication between the pre-amplifier 16 and the monitor 14 is activated or becomes available. In certain embodiments, as the QSM 68 of the pre-amplifier 16 fills up, the digital data may be transmitted to the QSM 68 of the patient monitor 14, which may then be downloaded to the RAM 52. In an embodiment, there may be multiple parallel paths for separate amplifiers, filters, and A/D converters for multiple light wavelengths or spectra received by the pre-amplifier 16, such as if detector signals are received from one or more sensors 12. In certain embodiments, the signal processing circuitry 58 may alternatively or additionally be included within the patient monitor 14.

In an embodiment, based at least in part upon the received signals corresponding to the light received by detector 20, the processor 46 may calculate the oxygen saturation using various algorithms. These algorithms may use coefficients, which may be empirically determined. For example, algorithms relating to the distance between an emitter 16 and various detector elements in a detector 20 may be stored in a ROM 52 and accessed and operated according to processor 48 instructions.

Furthermore, one or more functions of the monitor 14 may also be implemented directly in the pre-amplifier 16. For example, in some embodiments, the pre-amplifier 16 may include one or more processing components capable of calculating the physiological characteristics from the signals obtained from the patient 41. In accordance with the present techniques, the sensor 12 may be configured to provide optimal contact between a patient and the detector 20, and/or the emitter 16. The sensor 12 may have varying levels of processing power, and may output data in various stages to the pre-amplifier 16 via the cable 22. For example, in some embodiments, the data output to the pre-amplifier 16 may be analog signals, such as detected light signals (e.g., pulse oximetry signals or regional saturation signals), or processed data.

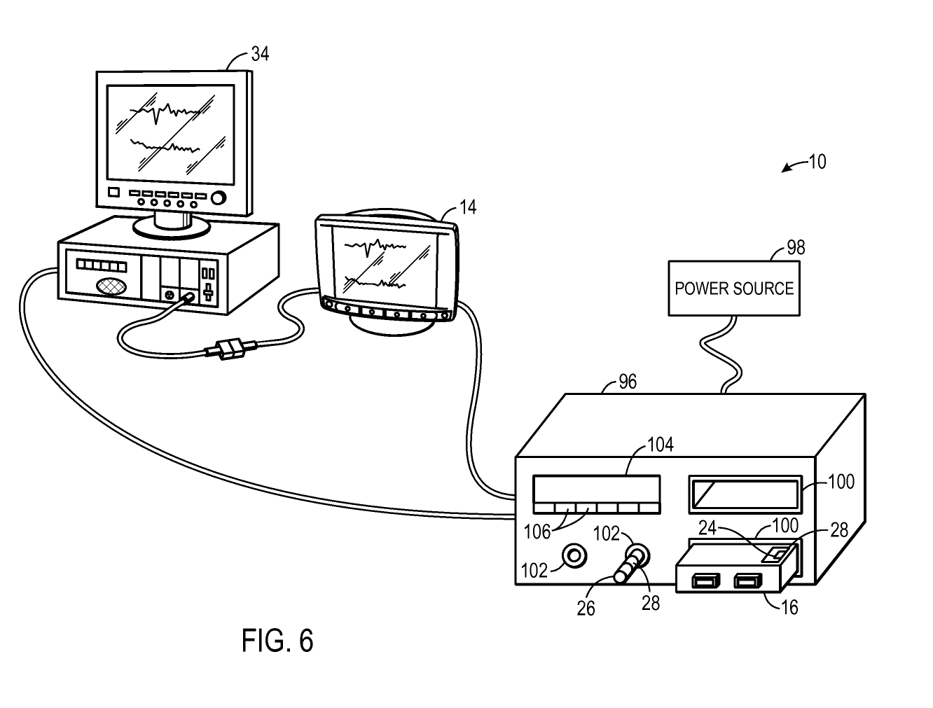

In certain embodiments, the pre-amplifier 16 may include an energy storage device 69. The energy storage device 69 may be a power source configured to supply power to the wireless pre-amplifier 16. In certain embodiments, the energy storage device 69 may be a battery source (e.g., rechargeable battery), or may be a storage device configured to recharge from an external source (e.g., a linking/charging device as further described with respect to FIG. 6). In certain embodiments, the energy storage device 69 may be coupled to an external wall-outlet, and may be configured to receive AC power that may be directly used and/or stored for future use.

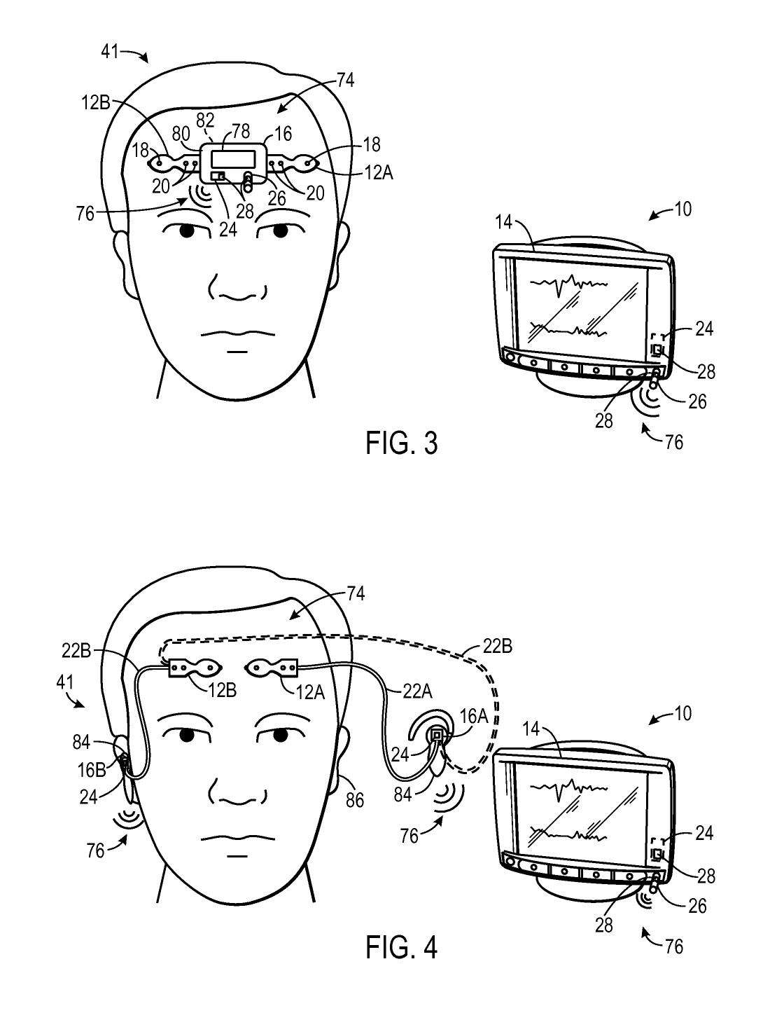

FIG. 3 is a perspective view of an embodiment of the monitoring system 10 of FIG. 1, illustrating the wireless pre-amplifier 16 and the sensor 12 applied to a forehead 74 of the patient 41. In some embodiments, the wireless pre-amplifier 16 may be sized such that it is suitable to be applied to the forehead 74. For example, the physical parameters (e.g., size, length, width, weight, etc.) of the pre-amplifier 16 may be configured such that it may be comfortably applied to the patient's forehead 74. In the illustrated embodiment, the wireless pre-amplifier 16 may be coupled to one or more sensors 12 (e.g., a first sensor 12A and a second sensor 12B) via the cable 22 (not shown), and may be configured to receive the signals collected by the sensors 12 via the cable 22. As noted above, the pre-amplifier 16 may be configured for amplifying, filtering, and digitizing the electrical signals received from the detectors 20 before transmitting the processed signals (e.g., digital data) to the patient monitor 14.

In particular, the wireless pre-amplifier 16 may include the wireless module 24 and/or the wireless attachment 26 for establishing a wireless communications channel 76 between the pre-amplifier 16 and the monitor 14. The wireless communications channel 76 may allow remote monitoring of the patient 74. For example, the wireless pre-amplifier 16 may allow the patient 74 to freely move in a location remote from the patient monitor 14 without compromising an operator's ability to monitor and assess the patient's current physiological condition. In addition, the wireless attachment 26 may provide wireless connectivity to devices without altering hardware of the components of the system 10.

In certain embodiments, as noted above, the wireless pre-amplifier 16 or the patient monitor 14 includes the indicator feature 28 on the wireless modules 24 or the wireless attachments 26. The system 10 and/or the operator may utilize the indicator feature 28 to visualize the pairing and/or the status of the wireless connectivity between the pre-amplifier 16 and the patient monitor 14. For example, indicator feature 28 may be a speaker for emitting audible indicators (e.g., alarms, voice warnings, beep tones), possibly with various frequencies, pitches, and/or volume amplitudes, indicator lights (e.g., LEDs, different flashing patterns of LEDs, different LED colors, color changing bands, color patterns or schemes, etc.), and so forth. The indicator feature 28 may assist the operator or the patient 30 quickly and safely in identify or locate the coupling or pairing between the patient monitor 12 and the wireless pre-amplifier 16. In certain embodiments, the wireless pre-amplifier 16 may include a display screen 78 (e.g., electronic ink display) disposed on a front surface 80. The pre-amplifier 16 may be configured to display the connectivity status of the pre-amplifier 16 and the monitor 14, may display warnings to the operator when the pre-amplifier 16 is coupled (or not coupled) to the monitor 12, may display warnings to the operator when wireless connectivity is lost between the pre-amplifier 16 and the monitor 14, may provide an indication of strength of connection, or may display information to help identify the location of the one or more wireless sensors 14 coupled to the pre-amplifier 16, and so forth.

In some embodiments, the pre-amplifier 16 may include various attachment features configured to help secure the pre-amplifier 16 in a desired location. For example, in the illustrated embodiment, the pre-amplifier 16 may include a patient contacting adhesive layer 82 laminated on a bottom surface (not shown) of the pre-amplifier 16, where the bottom surface is opposite to the front surface 80. The patient-contacting adhesive layer 82 may include any adhesive material suitable for integration into medical devices (e.g., a hypoallergenic adhesive material). In some embodiments, the adhesive material may be substantially transparent with respect to the wavelengths of light, such that the adhesive material does not interfere with the function of the sensors 12. In certain embodiments, other types of attachment features may be used to securely apply the pre-amplifier 16 to the tissue of the patient, and to secure the placement of the pre-amplifier 16 with respect to the sensors 12. In the illustrated embodiment, the pre-amplifier 16 is disposed between two sensors 12 on the forehead 74 of the patient 41. In other embodiments, the pre-amplifier 16 may be disposed on any part of the patient's head, and may be coupled to any number of sensors 12 via the cable 22.

Further, in certain embodiments, the pre-amplifier 16 may be disposed on a structure applied proximate the head of the patient 41, as illustrated with respect to FIG. 4. For example, FIG. 4 is a perspective view of an embodiment of the monitoring system 10 of FIG. 1, illustrating the wireless pre-amplifier 16 and the sensor 12 coupled to an ear-piece 84 attached to an ear 86 of the patient 41. In some embodiments, the wireless pre-amplifier 16 may be sized such that it is suitable to be attached to the ear-piece 84. For example, the physical parameters (e.g., size, length, width, weight, etc.) of the pre-amplifier 16 may be configured such that it may fit on a portion of the ear-piece 84 that is worn by the patient 41.

In the illustrated embodiment, the wireless pre-amplifiers 16 may be coupled to one or more sensors 12 (e.g., the first sensor 12A and the second sensor 12B) via the cables 22. For example, the first sensor 12A may include a first cable 22A configured to transmit signals collected by the first sensor 12A to a first pre-amplifier 16A. As noted above, the first pre-amplifier 16A may be configured to receive the signals collected by the sensor 12A via the cable 22A. Likewise, the second sensor 12B may include a second cable 22B configured to transmit signals collected by the second sensor 12B to a second pre-amplifier 16B. In the illustrated embodiment, the cables 22 may be of a suitable length to extend from the sensors 12 on the forehead 74 of the patient 41 to the ear 86 of the patient 41. Further, as noted above, the pre-amplifiers 16 may be configured for amplifying, filtering, and digitizing the electrical signals received from the detectors 20 on the sensors 12 before transmitting the processed signals (e.g., digital data) to the patient monitor 14 via the wireless communications channel 76. In certain embodiments, each sensor 12 may be communicatively coupled to a respective pre-amplifier 16, while in other embodiments, one or more sensors 12 disposed on the forehead 74 are communicatively coupled to a single pre-amplifier 16. For example, in certain embodiments, both the first sensor 12A and the second sensor 12B may be communicatively coupled to the first pre-amplifier 16A via the first and second cables 22A and 22B, respectively.

As noted above, in certain embodiments, the wireless pre-amplifier 16 may include the wireless module 24 and/or the wireless attachment 26 for establishing the wireless communications channel 76 between the pre-amplifier 16 and the monitor 14. In other embodiments, the pre-amplifier 16 may be communicatively coupled (e.g., attached) to a device configured with wireless communications. For example, the pre-amplifier 16 may utilize the wireless capabilities of the ear-piece 84 to communicate with the patient monitor 14 on the wireless communications channel 76. It should be noted that the ear-piece 84 may utilize any suitable wireless protocol, as described above with respect to FIG. 1.

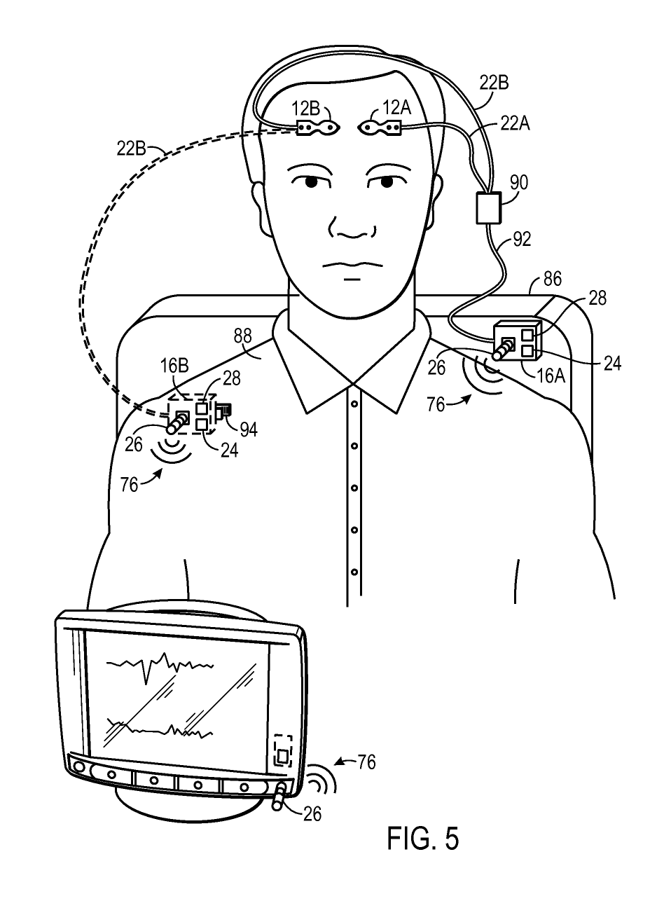

In certain embodiments, the pre-amplifier 16 may be disposed proximate to the patient's body, as further described with respect to FIG. 5. For example, FIG. 5 is a perspective view of an embodiment of the monitoring system 10 of FIG. 1, illustrating the wireless pre-amplifier 16 and the sensor 12 disposed proximate to the patient and remote from the patient monitor 14. In some embodiments, the wireless pre-amplifier 16 may be sized such that it is suitable to be placed proximate to the patient's body, such as on a surface 86 (e.g., a pillow, a desk, a side table, a hospital bed, a medical device, etc.) or a material 88 worn by the patient (e.g., a shirt, an armband, a belt, a wristlet, a cap, a hospital accessory, any article of clothing, etc.). For example, the physical parameters (e.g., size, length, width, weight, etc.) of the pre-amplifier 16 may be configured such that it may be comfortably attached to the surface 86 or the material 88 proximate to the patient's body.

In the illustrated embodiment, the wireless pre-amplifiers 16 (e.g., the first pre-amplifier 16A and the second pre-amplifier 16B) may be coupled to one or more sensors 12 (e.g., the first sensor 12A and the second sensor 12B) via the cables 22 (e.g., the first cable 22A and the second cable 22B). For example, the first sensor 12A may utilize the first cable 22A to transmit signals collected by the first sensor 12A to a first pre-amplifier 16A. In certain embodiments, the second sensor 12B may utilize the second cable 22B to transmit signals collected by the second sensor 12B to a second pre-amplifier 16B. In some embodiments, the second sensor 12B may be configured to transmit signals to the first pre-amplifier 16A (as described with respect to FIG. 4), or the second cable 22B may be configured to be coupled through a connector 90 to a patient interface cable 92, which in turn may be coupled to the first pre-amplifier 16A. In the illustrated embodiment, the cables 22, 90, and 92 may be of a suitable length to extend from the sensors 12 on the forehead 74 of the patient 41 to a location proximate the patient's body, such as on the surface 86 proximate the patient's body or the material 88 on the patient's body.

In certain embodiments, the pre-amplifier 16 may include various attachment features configured to help secure the pre-amplifier 16 in a desired location, such as on the material 88 on the patient's body. For example, an attachment feature 94 may be a hook, a clip, a pin, a fastener, a button, a tape, or any other feature configured to attach the pre-amplifier 16 to the material 88. In some embodiments, the attachment feature 94 may be a stand, an adhesive, a velcro clip, or any other feature configured to secure the pre-amplifier 16 to the surface 86.

In certain embodiments, the patient monitoring 10 of FIG. 1 may include additional components coupled to the patient monitor 14 and/or the multi-parameter patient monitor 34. For example, FIG. 6 is a block diagram of an embodiment of the monitoring system 10 of FIG. 1, illustrating a charging/linking station 96 configured to charge and/or pair one or more wireless pre-amplifiers 16 with one or more wireless attachments 26. In certain embodiments, the system 10 may include the charging/linking station 96 communicatively coupled to a power source 98, the patient monitor 14, and/or the multi-parameter patient monitor 34. In certain embodiments, the charging/linking station 96 may be configured to couple and/or pair (e.g. link) one or more pre-amplifiers 16 with one or more wireless attachments 26. Once paired, the wireless attachment 26 may then be attached to a pre-existing component or device of the system 10 (e.g., the patient monitor 14) to provide wireless connectivity between the device of the system 10 attached to the wireless attachment 26 and the pre-amplifier 16. It should be noted that the charging/linking station 96 may be configured to charge and/or pair any two devices, such pairing two wireless pre-amplifiers 16, pairing two wireless attachments 26, pairing two wireless attachments 26 with one pre-amplifier 16, etc.

For example, the charging/linking station 96 may include one or more pre-amplifier slots 100, one or more wireless attachment slots 102, and a display 104. The pairing or coupling may be activated by inserting one or more pre-amplifiers 16 into the pre-amplifier slots 100 at the same time as (or within a reasonable time period as) one or more wireless attachments 26 are inserted into the wireless attachment slots 102. In certain embodiments, the pre-amplifiers 16 and/or the wireless attachments 26 may include a unique identifier (e.g., a patient ID number, a patient name, a unique bar code number, a unique serial number, a patient identification tag or bracelet, etc.) that are recognized and displayed by the charging/linking station 96 on the display 104. The charging/linking station 96 may also include various input components 106, such as knobs, switches, keys and keypads, buttons, etc., to provide for operation and configuration of the station 96. Accordingly, in certain embodiments, the charging/linking station 96 may accurately pair or link the desired devices after authentication by a user/operator via user inputs and the unique identifiers. In other embodiments, the charging/linking station 96 may be configured to automatically pair any two or more inserted devices. In such embodiments, the indicator features 28 may be utilized to visualize the pairing and/or the status of the pairing.

It should be noted that in certain embodiments, the pairing or linking between two components of the system 10 may be done with other techniques known to one skilled in the art. For example, the embodiments herein may utilize one or more physical pairing techniques (e.g., electrical features, polarized magnets, etc.) for coupling the wireless pre-amplifier 16 to the wireless attachment 26 and/or the patient monitor 14 via physical contact. In other embodiments, the wireless sensors described herein may use one or more unique tokens to establish a coupling between the wireless pre-amplifier 16 to the wireless attachment 26 and/or the patient monitor 14. In such embodiments, each unique token may be any card, paper, or plastic that has a unique identifying feature that identifies the wireless pre-amplifier 16, the wireless attachment 26, and/or the patient monitor 14 to be coupled. In certain embodiments, the tokens may be inserted into the linking/charging device 96 to pair the components.

In particular, once paired, the one or more wireless attachment 26 may be attached to the patient monitor 14 to establish a wireless communications channel between the patient monitor 14 and the previously paired pre-amplifier 16. As noted above, in certain embodiments, the system 10 and/or the operator may utilize the indicator feature 28 to visualize the pairing and/or the status of the wireless connectivity between the pre-amplifier 16 and the patient monitor 14. In particular, it may be beneficial to pair or couple the pre-amplifier 16 with the patient monitor 14 so that patient monitor 14 is displaying information from the intended source to safely and accurately identify the patient being monitored when viewing physiological information on a patient monitor. In should be noted that in certain embodiments, the charging/linking station 96 may additionally pair or link the wireless module 24 of the pre-amplifier 16 with the wireless attachment 26. Accordingly, the charging/linking station 96 may enable wireless connectivity between devices not configured with a wireless module 24 (such as a patient monitor 14 without preexisting wireless capabilities) and devices configured with a wireless module 24 (such as a pre-amplifier 16 having built-in wireless capabilities) via the wireless attachment 26. Accordingly, the wireless attachment 26 may be configured to provide wireless connectivity between any component of the system 10 linked with cables, such as for example, between the multi-parameter monitor 34 and the patient monitor 14.

In certain embodiments, the charging/linking station 96 may be coupled to a power source 98. The power source 98 may be a battery source (e.g., rechargeable battery) or may be a wall-outlet configured to deliver electrical power to the charging/linking station 96, and the components inserted into the charging/linking station 96. In particular, each of the components inserted into the charging/linking station 96 (e.g., the pre-amplifier 16, the wireless attachment 26) may include a rechargeable battery source, which in some embodiments may be a user-removable battery charged externally from the charging/linking station 96. Additionally, the power source 98 may include AC power, provided by an electrical outlet, and the power source 98 may be connected to the AC power via a power adapter through a power cord (not shown). This power adapter may also be used to directly recharge one or more batteries of the power source 98 and/or to power the charging/linking station 96.

What is claimed is:

1. A patient monitoring system, comprising:
a wireless pre-amplifier configured to wirelessly communicate with a patient monitor, the wireless pre-amplifier comprising:
one or more sensor ports configured to receive a signal related to a physiological parameter of a patient from one or more sensors applied to the patient;
a first wireless attachment configured to wirelessly transmit the signal related to the physiological parameter of the patient to the patient monitor over a wireless communications channel, wherein the first wireless attachment is configured to be physically and electrically coupled to the wireless pre-amplifier; and
an indicator feature on the first wireless attachment configured to provide a user perceptible indication related to a status of wireless communication over the wireless communication channel between the wireless pre-amplifier and the patient monitor.

2. The patient monitoring system of claim 1, wherein the patient monitoring system comprises a regional oximetry monitoring system, the patient monitor comprises a regional oximetry monitoring system, and the wireless medical sensor comprises a regional oximetry sensor.

3. The patient monitoring system of claim 1, wherein the first wireless attachment is an electrical connector configured to wirelessly transmit and receive the signal related to the physical parameters.

4. The patient monitoring system of claim 1, wherein the electrical connector is at least one of a 9-pin connector, a 15-pin connector, a 24-pin connector, a 26-pin connector, a medi-snap connector, or a serial connector.

5. The patient monitoring system of claim 1, comprising the patient monitor, wherein a second wireless attachment is configured to be physically and electrically coupled to the patient monitor and is operatively paired with the first wireless attachment on the wireless pre-amplifier.

6. The patient monitoring system of claim 1, comprising the patient monitor, wherein the patient monitor comprises a wireless module configured to operatively pair the patient monitor to the first wireless attachment on the wireless pre-amplifier. The patient monitoring system of claim 1, wherein the indicator feature comprises at least one of an LED light, a LED flashing pattern, a LED color scheme, or a color changing band, or a combination thereof.

7. The patient monitoring system of claim 1, comprising a linking station configured to operatively pair the first wireless attachment of the wireless pre-amplifier to the patient monitor.

8. The patient monitoring system of claim 1, comprising a third wireless attachment configured to be physically and electrically coupled to another component of the patient monitoring system.

9. The patient monitoring system of claim 8, wherein the component of the patient monitoring system comprises a multi-parameter patient monitor.

10. A patient monitoring system, comprising:
a wireless pre-amplifier configured to obtain a signal related to a physiological parameter from a patient from one or more sensors applied to the patient, the wireless pre-amplifier comprising a first wireless module configured to wirelessly transmit and receive data; and
a patient monitor configured to wirelessly transmit to and receive data from the wireless pre-amplifier via a second wireless module.

11. The patient monitoring system of claim 10, wherein the patient monitoring system comprises a regional oximetry monitoring system, the patient monitor comprises a regional oximetry monitoring system, and the wireless medical sensor comprises a regional oximetry sensor.

12. The patient monitoring system of claim 10, wherein the second wireless module is an electrical connector configured to wirelessly transmit and receive data, wherein the electrical connector is configured to physically and electrically couple to an input port on the patient monitor.

13. The patient monitoring system of claim 10, comprising a linking station configured to operatively pair the first wireless module with the second wireless module.

14. The patient monitoring system of claim 10, wherein the wireless pre-amplifier is configured to be disposed on the patient's forehead proximate to one or more sensors applied to the patient.

15. The patient monitoring system of claim 10, wherein the wireless pre-amplifier is configured to be physically coupled to an ear-piece configured to wirelessly transmit to and receive data from the patient monitor.

16. The patient monitoring system of claim 10, wherein the wireless pre-amplifier is configured to be disposed on a material or a surface proximate to the patient's body, wherein the material is an article of clothing disposed on the patient, and wherein the surface is any surface not disposed on the patient's body.

17. A patient monitoring system, comprising:
a wireless pre-amplifier configured to obtain a signal related to a physiological parameter from a patient from one or more sensors applied to the patient, the wireless pre-amplifier comprising a first wireless module configured to wirelessly transmit and receive data;
a patient monitor configured to wirelessly transmit to and receive data from the wireless pre-amplifier via a second wireless module; and
a linking station configured to operatively pair the first wireless module with the second wireless module.

18. The patient monitoring system of claim 17, wherein the linking station is configured to receive a unique identifier, a patient ID number, a patient name, a unique bar code number, a unique serial number, or a patient identification tag, or any combination thereof, from the first or second wireless modules, and wherein the linking station is configured to utilize the unique identifier to operatively pair the first and the second wireless modules.

19. The patient monitoring system of claim 18, wherein the linking station comprises a display configured to display information related to the pairing of the first and second wireless modules.

20. The patient monitoring system of claim 18, wherein the second wireless module is an electrical connector configured to wirelessly transmit and receive data, wherein the electrical connector is configured to physically and electrically couple to an input port on the patient monitor.
